(19) United States
(12) Patent Application Publication
(10) Pub. No.: US 2011/0260100 A1
(43) Pub. Date: Oct. 27, 2011

(54) Novel Metal-Organic Frameworks as Electrode Material for Lithium Ion Accumulators

(75) Inventors: Natalia Trukhan, Ludwigshafen (DE); Ulrich Müller, Neustadt (DE); Alexander Panchenko, Ludwigshafen (DE); Itamar Michael Malkowsky, Speyer (DE); Andreas Fischer, Heppenheim (DE)

(73) Assignee: BASF SE, Ludwigshafen (DE)

(21) Appl. No.: 13/088,865

(22) Filed: Apr. 18, 2011

Related U.S. Application Data
(60) Provisional application No. 61/326,256, filed on Apr. 21, 2010.

(51) Int. Cl. H01M 4/60; C07C 63/14; H01M 4/24

(52) U.S. Cl. 252/182.1; 562/480

(57) ABSTRACT

Described is an electrode material which is suitable for a lithium ion accumulator and comprises a porous metal-organic framework, wherein the framework comprises lithium ions and optionally at least one further metal ion and at least one bidentate organic compound and the at least one bidentate organic compound is based on a dihydroxydicarboxylic acid which can be reversibly oxidized to a quinoid structure. Also described is a porous metalorganic framework, the use thereof and also lithium ion accumulators comprising such electrode materials.

### FIELD

The present invention relates to electrode materials which are suitable for a lithium ion accumulator and comprise a porous metal-organic framework, the metal-organic framework as such, the use thereof and also accumulators comprising the electrode material.

### BACKGROUND

Lithium ion batteries or lithium ion accumulators have a high energy density and are thermally stable. Here, the fact that a high cell voltage can be obtained when using lithium because of its high negative standard potential is exploited.

However, the high reactivity of elemental lithium requires the provision of special lithium sources and electrolytes.

In a relatively recent development, porous metal-organic frameworks which comprise lithium ions and are thus in principle suitable for lithium ion batteries or accumulators are described. Thus, for example, G. de Combarieu et al., Chem. Mater. 21 (2009), 1602-1611, describes the electrochemical suitability of a porous metal-organic framework based on iron terephthalate in lithium ion batteries.

Further Li/Fe-based metal-organic frameworks having reversible redox properties and sorption properties are described by G. Ferey et al., Angewandte Chemie 119 (2007), 3323-3327. Here too, terephthalic acid serves as organic ligand in the metal-organic framework.

Despite the electrode materials based on metal-organic frameworks which are known from the prior art for lithium ion batteries, there is still a need for improved systems in respect of suitability as electrode material, in particular with regard to the electrochemical capacity thereof (very particularly based on the mass.

### SUMMARY

Embodiments of the invention provide an electrode material which is suitable for a lithium ion accumulator and comprises a porous metal-organic framework, wherein the framework comprises lithium ions and optionally at least one further metal ion and at least one bidentate organic compound and the at least one bidentate organic compound is based on a dihydroxydicarboxylic acid which can be reversibly oxidized to a quinoid structure.

A further aspect of the present invention is a porous metal-organic framework as set forth here.

### DETAILED DESCRIPTION

It has been found that the use of a dihydroxydicarboxylic acid which can be reversibly oxidized to a quinoid structure or a derivative thereof enables frameworks which are particularly suitable for lithium ion accumulators and have good capacity/mass values to be provided.

The porous metal-organic framework of the invention comprises, firstly, lithium ions. The lithium ions can here be partly bound, in particular ionically, to deprotonated hydroxyl functions. Lithium ions can also serve to make up the skeleton of a framework. In this case, it is sufficient for only lithium ions to be present in the framework.

In addition, one or more metal ions other than lithium can optionally be present. These then participate in formation of the metal-organic framework. Thus, for example, a further metal ion can be present in addition to lithium ions. It is likewise possible for two, three, four or more than four further metal ions to be present. Here, the metal ions can be derived from one metal or various metals. If at least two metal ions are derived from one and the same metal, these have to be present in different oxidation states.

In a preferred embodiment, the porous metal-organic framework of the invention comprises no further metal ions in addition to lithium ions.

In an alternative embodiment, the porous metal-organic framework of the invention comprises at least one further metal ion in addition to lithium ions. The at least one further metal ion is preferably selected from the group consisting of the metals cobalt, iron, nickel, copper, manganese, chromium, vanadium and titanium. Greater preference is given to cobalt, iron, nickel and copper. Cobalt and copper are even more preferred.

At least one bidentate organic compound is necessary to build up the porous metal-organic framework of the invention. It is therefore possible for either one at least bidentate organic compound or a plurality of different at least one bidentate organic compounds to be present. Thus, two, three, four or more different at least one bidentate organic compounds can be present in the porous metal-organic framework of the invention.

The at least one bidentate organic compound is based on a dihydroxydicarboxylic acid which can be reversibly oxidized to a quinoid structure.

In this context, “quinoid” means, in particular, that the two hydroxy groups can be oxidized to oxo groups. “Reversibly” means, in particular, that, after reduction, the oxidation can be carried out again.

For the purposes of embodiments of the present invention, the term “derived” means that the at least one bidentate organic compound is present in partially or completely deprotonated form in respect of the carboxy functions. Furthermore, it is preferred that the at least one bidentate organic compound is also at least partially deprotonated in the reduced state in respect of its hydroxy groups and binds lithium ions, usually via an ionic bond. Furthermore, the term “derived” means that the at least one bidentate organic compound can have further substituents. Thus, one or more independent substituents such as amino, methoxy, halogen or methyl groups can be present in addition to the carboxyl function. Preference is given to no further substituents or only F substituents being present. For the purposes of the present invention, the term “derived” also means that the carboxyl function can be present as a sulfur analogue. Sulfur analogues are —C(═O)SH and the tautomer thereof and —C(S)SH. Preference is given to no sulfur analogues being present.

In addition to these at least bidentate organic compounds, the metal-organic framework can also comprise one or more monodentate ligands.

The at least one bidentate organic compound has to have a parent molecule which is capable of forming the quinoid system. This is achieved, in particular, by the parent molecule having a double bond system conjugated with the oxo groups, in particular by the presence of C—C double bonds. Such parent molecules are known to 30 those skilled in the art. Examples are benzene, naphthalene, phenanthrene or similar parent molecules. These then bear at least the hydroxy/hydroxide groups and the carboxy/carboxylate groups.

In a preferred embodiment, the dihydroxydicarboxylic acid is a dihydroxybenzenedicarboxylic acid, in particular 2,5-dihydroxyterephthalic acid.

The porous metal-organic frameworks of the invention can in principle be prepared in the same way as comparable metal-organic frameworks which are known from the prior art. In particular, reference may here be made to lithium-based metal-organic frameworks as described in WO-A 2010/012715.

The preparation of doped or impregnated metal-organic frameworks is described, for example, in EP-B 1 785 428 and EP-A 1 070 538. Apart from the conventional method of preparing the porous metal-organic frameworks (MOFs) as described, for example, in U.S. Pat. No. 5,648,508, these can also be prepared by an electrochemical route. In this 5 respect, reference is made to DE-A 103 55 087 and WO-A 2005/049892. The metal-organic frameworks prepared by this route have particularly good properties.

A further aspect of the present invention is an accumulator comprising the electrode material of the invention.

The production of accumulators according to the invention is known in principle from the prior art for the production of lithium ion accumulators or lithium ion batteries. Here, reference may be made, for example, to DE-A 199 16 043. Since the structural principle for accumulators and batteries is the same in this respect, reference will hereinafter be made to a lithium ion battery or battery in the interest of simplicity.

The electrode material which is suitable for the reversible storage of lithium ions is usually fixed to power outlet electrodes by means of a binder.

In the charging of the cell, electrons flow through an external voltage source and lithium cations flow through the electrolyte to the anode material. When the cell is utilized, the lithium cations flow through the electrolyte while the electrons flow through a load from the anode material to the cathode material.

To avoid a short circuit within the electrochemical cell, an electrically insulating layer through which lithium cations can nevertheless pass is present between the two electrodes. This can be a solid electrolyte or a conventional separator.

In the production of many electrochemical cells, e.g. in the case of a lithium ion battery in the form of a round cell, the required battery foils/films, i.e., cathode foils, anode foils and separator foils, are combined by means of a rolling device to form a battery roll. In the case of conventional lithium ion batteries, the cathode and anode foils are connected to power outlet electrodes in the form of, for example, an aluminum or copper foil. Such metal foils ensure sufficient mechanical stability.

The separator film, on the other hand, must on its own withstand the mechanical stresses, which in the case of conventional separator films based on, for example, polyolefins in the thickness used does not present a problem.

The present invention further provides for the use of a porous metal-organic framework according to the invention in an electrode material for lithium ion accumulators. The electrode material of the invention is particularly suitable for use in an accumulator. The electrode material can basically be used in electrochemical cells.

The present invention therefore further provides an electrochemical cell comprising an electrode material according to the invention and also provides for the use of a porous metal-organic framework according to the invention in an electrode material for electrochemical cells.

### BRIEF DESCRIPTION OF THE DRAWINGS

FIG. 1: XRD analysis of an Li-2,5-dihydroxyterephthalic acid MOF. Here, as in FIGS. 3 to 5, the intensity I (Lin(Counts)) is shown as a function of the 2 theta scale (2Θ).

FIG. 2: SEM analysis of an Li-2,5-dihydroxyterephthalic acid MOF.

FIG. 3: XRD analysis of an Li—Co-2,5-dihydroxyterephthalic acid MOF.

FIG. 4: XRD analysis of a Co-2,5-dihydroxyterephthalic acid MOF.

FIG. 5: XRD analysis of a Cu-2,5-dihydroxyterephthalic acid MOF.

FIG. 6: SEM analysis of a Cu-2,5-dihydroxyterephthalic acid MOF.

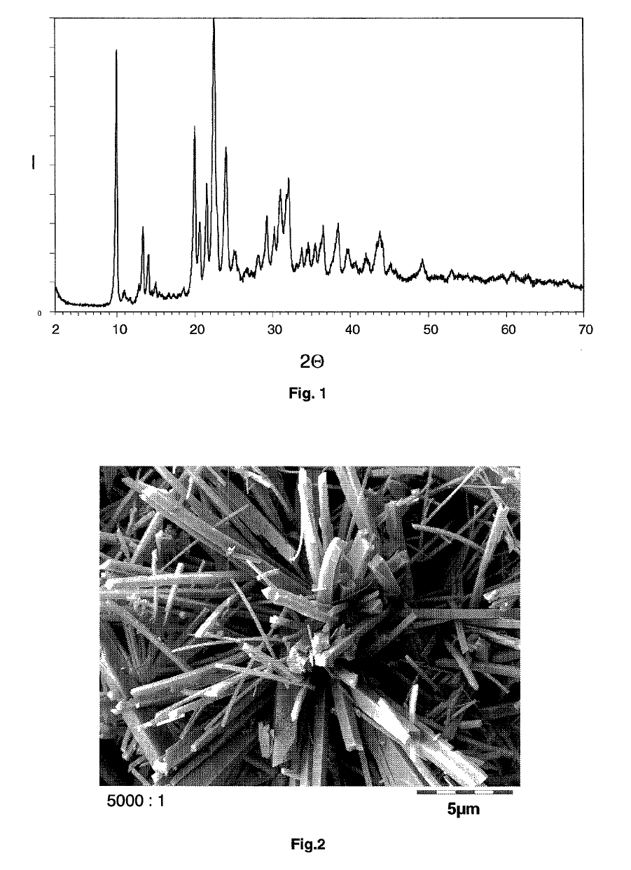

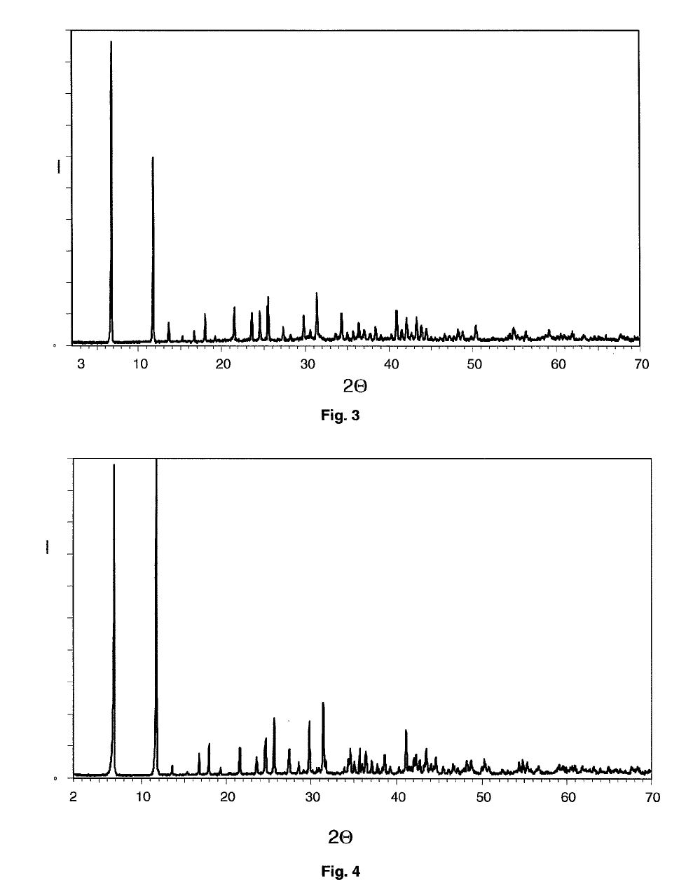

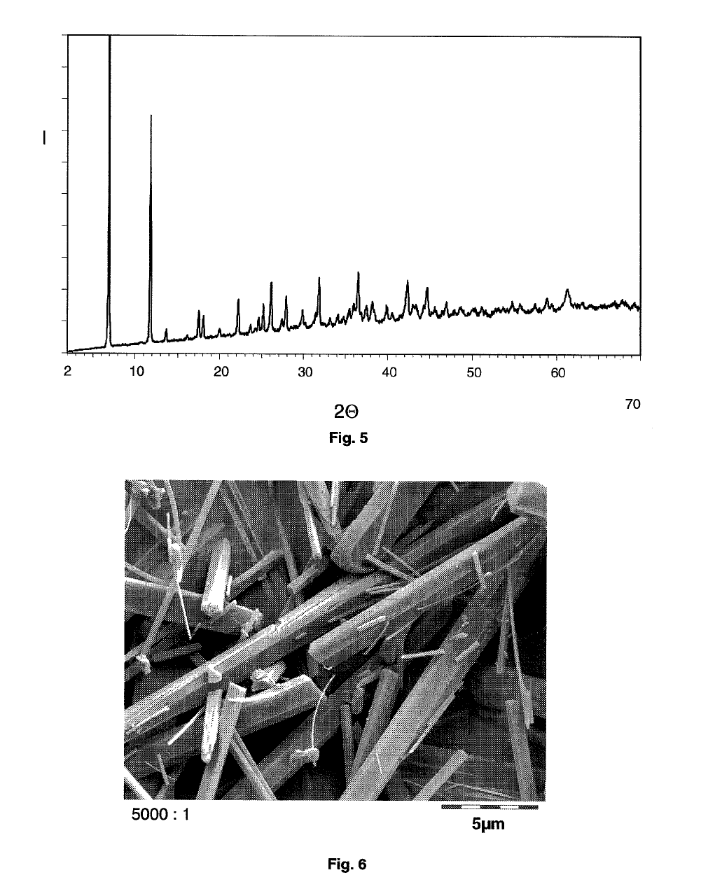

### EXAMPLES

### Example 1

### Synthesis of an Li-2,5-dihydroxyterephthalic acid MOF

### Experimental Method:

| Starting material | Mol | | Calculated | | Experimental | |
|---|---|---|---|---|---|---|
| 1) 2,5-Dihydroxyterephthalic | 151.5 | mmol | 30.0 | g | 30.0 | g |
| acid | | | | | | |
| 2) Lithium hydroxide | 606.0 | mmol | 14.3 | g | 14.3 | g |
| 3) DMF | 8.17 | mol | 600.0 | g | 600.0 | g |
| 4) Water | 11.6 | mol | 210.0 | g | 210.0 | g |

In a glass beaker, the 2,5-dihydroxyterephthalic acid is dissolved in DMF. In a second glass beaker, the lithium hydroxide is dissolved in water. This solution is slowly added dropwise to the first yellow solution. Shortly before the end of the addition, the solution becomes turbid and changes into a green suspension. This is filtered after 1 hour and the solid is washed 4 times with 100 ml each time of DMF. The filtercake is dried overnight at RT under reduced pressure.

Product weight: 35.9 g
Color: yellowish green
Solids concentration: 4.2%
Yield based on Li: 77.9%

### Analyses:

Langmuir SA (preactivation at 130° C.): 13 m²/g (BET: 9 m²/g)

### Chemical Analysis:

| | |
|---|---|
| Carbon | 42.1 g/100 g |
| Oxygen | 41.1 g/100 g |
| Nitrogen | 4.7 g/100 g |
| Li | 9.0 g/100 g |

### Example 2

### Li Doping of a Co-2,5-dihydroxyterephthalic acid MOF (Co-DHBDC MOF)

### Experimental Method:

| Starting material | Mol | | Calculated | | Experimental | |
|---|---|---|---|---|---|---|
| 1) Co-DHBDC MOF | 5.0 | g | 5.0 | g | | |
| 2) Lithium hydroxide | 25 | mmol | 0.6 | g | 0.6 | g |
| 3) DMF | 1.09 | mol | 80.0 | g | 80.0 | g |
| 4) Water | 0.5 | mol | 9.0 | g | 9.0 | g |

In a- glass beaker, the Co-2,5-dihydroxyterephthalic acid MOF (see 2a) is suspended in DMF. In a second glass beaker, the lithium hydroxide is dissolved in water. This 25 solution is added dropwise to the first red suspension. The suspension becomes slightly dark red. After 2 hours, the suspension is filtered and the solid is washed 4 times with 100 ml each time of DMF. The filtercake is dried overnight at RT under reduced pressure and subsequently at 130° C. for 16 hours under reduced pressure.

Product weight: 5.5 g
Color: brownish green
Solids concentration: 5.8%
Yield based on Li: 88%

### Analyses:

Langmuir SA (preactivation at 130° C.): 169 m²/g (BET: 125 m²/g)

### Chemical Analysis:

| | |
|---|---|
| Carbon | 32.0 g/100 g |
| Oxygen | 37.4 g/100 g |
| Nitrogen | 5.1 g/100 g |
| Co | 21.1 g/100 g |
| Li | 2.8 g/100 g |

### Example 2a

### Synthesis of a Co-2,5-dihydroxyterephthalic acid MOF

### Starting Materials:

1) 64.85 g of Co(NO₃)₂×6 H20
2) 33.25 g of 2,5-dihydroxyterephthalic acid

### Solvents:

1) 3500 ml (3325 g) of DMF
2) 175 ml of H₂O

### Experimental Method

| | |
|---|---|
| a) Synthesis: | 2,5-Dihydroxyterephthalic acid and Co nitrate |
| | were dissolved in a 4 I flask, heated to 100° C. |
| | over a period of 1.5 hours and stirred at |
| | 100° C. under N2 for 8 hours |
| b) Work-up: | under N2 |
| | filtered at RT, washed with 1000 ml of |
| | DMF/2000 ml of MeOH |
| | filtrate halved and extracted with 600 ml in |
| | each case of MeOH overnight (16 h). |
| c) Drying: | over the weekend at RT under reduced |
| | pressure |

Color: orange

### Yield: 47.2 g

Solids concentration: 1.31%
Yield based on Co: 92.0%

### Analyses:

Langmuir SA (preactivation at 130° C.): 1311 m²/g (BET: 961 m²/g)

### Chemical Analysis:

| | |
|---|---|
| Carbon | 30.8 g/100 g |
| Co | 25.5 g/100 g |

### Example 3

### Li Doping of a Cu-2,5-dihydroxyterephthalic acid MOF (Cu-DHBDC MOF)

| Starting material | Mol | | Calculated | | Experimental | |
|---|---|---|---|---|---|---|
| 5) Cu-DHBDC MOF | | | 5.0 | g | 5.0 | g |
| 6) Lithium hydroxide | 80.8 | mmol | 0.6 | g | 0.6 | g |
| 7) DMF | 1.09 | mol | 80.0 | g | 80.0 | g |
| 8) Water | 0.5 | mol | 9.0 | g | 9.0 | g |

In a glass beaker, the CU-2,5-dihydroxyterephthalic acid MOF (see 3a) is suspended in DMF. In a second glass beaker, the lithium hydroxide is dissolved in water. This solution is added dropwise to the first suspension. After 2 hours, the suspension was filtered and the solid was washed 4 times with 100 ml each time of DMF. The filtercake is dried overnight at RT under reduced pressure and subsequently at 130° C. under reduced pressure for 16 hours.

Product weight: 5.5 9
Color: brown
Solids concentration: 5.8% by weight

### Analyses:

Langmuir SA (preactivation at 200° C.): 577 m²/g (BET: 430 m²/g)

### Chemical Analysis:

| | |
|---|---|
| Cu | 33.0 g/100 g |
| Li | 3.7 g/100 g |

### Example 3a

### Synthesis of a Cu-2,5-dihydroxyterephthalic acid MOF

### Starting Materials:

2×34.2 g of Cu(NO₃)₂×3 H₂0=2×141.6 mmol
   M=241.6 g/mol
2×13.3 g of 2,5-dihydroxyterephthalic acid=2×67.13 mmol
   M=198.13 g/mol

### Solvents:

2×700 ml of DMF, density: 0.95 g/ml=1300 g
2×35 ml of H₂O

### Experimental Method: 2×2 I Batches

### Synthesis:

2,5-dihydroxyterephthalic acid and Cu nitrate were dissolved in 2×2 I flasks, heated to 100° C. over a period of 1.5 hours and stirred at 100° C. for 8 hours

### Workup:

under N2
filtered at RT, washed with 2×250 ml of DMF/4×250 ml of MeOH residue extracted with 330 ml of MeOH overnight (16 h).
   Drying: 48 h at RT under reduced pressure
   Activation: 16 h at 130° C. under reduced pressure
   Color: reddish brown

### Yield: 40.7 g

Solids concentration: 2.8%
Metal analysis: Cu 39%

### Analyses:

Langmuir SA (preactivation at 130° C.): 1183 m²/g (BET: 879 m²/g)

### Chemical Analysis:

| | |
|---|---|
| Carbon | 26.3 g/100 g |
| Cu | 39 g/100 g |

### Electrochemical Characterization

1.5 g of MOF, 0.75 g of Super P (conductive carbon black additive, from Timcal), 0.12 g of KS 6 (conductive graphite additive, from Timcal), 0.75 g of PVDF (polyvinylidene fluoride) were mixed together in 50 ml of NMP(N-methyl-2-pyrrolidone) and stirred for 10 hours.

The dispersion was applied to AI foil by means of a doctor blade and dried at 120° C. under reduced pressure for 10 hours.

Testing of the electrochemical cell according to the invention

To characterize the composite electrochemically, an electrochemical cell was constructed. Anode: Li foil 50 μm thick, separator: Freundenberg 2190, from Freundenberg; cathode on AI foil with MOF as described above; electrolyte: EC (ethylene carbonate)/DEC(diethyl carbonate) 3: 7% by volume with lithium hexafluorophosphate (LIPF₆) 1 mol/l.

Charging and discharging of the cell were carried out at a current of 0.02 mA. The results are summarized in table 1.

TABLE 1

| MOF material | Potential window, V | Capacity, mAh/g of MOF |
|---|---|---|
| Example 1 | 1.5-4.8 | 240 |
| Example 2 | 1.5-4.8 | 175 |
| Example 3 | 1.5-4.8 | 260 |

1. An electrode material which is suitable for a lithium ion accumulator and comprises a porous metal-organic framework, wherein the framework comprises lithium ions and optionally at least one further metal ion and at least one bidentate organic compound and the at least one bidentate organic compound is based on a dihydroxydicarboxylic acid which can be reversibly oxidized to a quinoid structure.

2. The electrode material according to claim 1, wherein one or more further metal ions are comprised.

3. The electrode material according to claim 2, wherein the at least one further metal ion is selected from the group consisting of the metals cobalt, iron, nickel, copper, manganese, chromium, vanadium and titanium.

4. The electrode material according to claim 1, wherein the dihydroxydicarboxylic acid is a dihydroxybenzenedicarboxylic acid.

5. The electrode material according to claim 1, wherein the dihydroxydicarboxylic acid is 2,5-dihydroxyterephthalic acid.

6. A porous metal-organic framework as set forth in claim 1.

7. A method of using a porous metal-organic framework according to claim 6 in an electrode material for lithium ion accumulators.

8. An accumulator comprising an electrode material according to claim 1.

9. An electrochemical cell comprising an electrode material according to claim 1.

10. A method of using a porous metal-organic framework according to claim 6, in an electrode material for electrochemical cells.
